(19) United States
(12) Patent Application Publication
(10) Pub. No.: US 2005/0282877 A1
(43) Pub. Date: Dec. 22, 2005

(54) Method of decreasing inflammation in kidney transplantion using angiotensin receptor blockers

(76) Inventors: Bryan N. Becker, Verona, WI (US); Lynn M. Jacobson, Madison, WI (US); Debra A. Hullett, Oregon, WI (US); Jon A. Weidanz, Amarillo, TX (US); Vaughan P. Wittman, Amarillo, TX (US)

Correspondence Address: Joseph T. Leone;DeWitt Ross & Stevens S.C., 8000 Excelsior Drive, Madison, WI, 53717, US

(21) Appl. No.: 11/104,165

(22) Filed: Apr. 12, 2005

Related U.S. Application Data
(60) Provisional application No. 60/561,608, filed on Apr. 13, 2004.

(51) Int. Cl. A61K 31/4184

(52) U.S. Cl. 514/381

(57) ABSTRACT

Disclosed is a method of inhibiting production of IFN-Γ in patients having a transplanted organ. The method involves administering to the patient an amount of an angiotensin receptor-blocking compound, the amount being effective to inhibit production of IFN-Γ by T cells. The method can be used to treat inflammation involving an allograft, to treat chronic allograft nephropathy, and to treat other pathologies associated with allograft rejection.

### CROSS-REFERENCE TO RELATED APPLICATIONS

This application claims priority under 35 USC.§119(e) to provisional application Ser. No. 60/561,608, filed Apr. 13, 2004, which is incorporated herein by reference.

### FEDERAL FUNDING

This invention was made with United States government support awarded by the following agencies: NIH AIO49285. The United States has certain rights to this invention.

### REFERENCE TO CITATIONS

Complete bibliographical citations to the references cited herein can be found in the list preceding the Claims.

### FIELD OF THE INVENTION

The invention is directed to a method of decreasing the production of interferon-gamma (IFN-Γ) by lymphocytes during and after organ transplantation in general and kidney transplantation in particular by administering one or more compounds that block receptors for angiotensin I and/or angiotensin II.

### BACKGROUND

The renin-angiotensin-aldosterone system plays an integral role in the pathophysiology of hypertension by affecting the regulation of fluid volume, electrolyte balance, and blood volume. Renin is produced by the kidneys and catalyzes the conversion of angiotensinogen into angiotensin I (Ang I). Angiotensin-converting enzyme (ACE) then converts Ang I into the highly active angiotensin II (Ang II). Ang II is a potent vasoconstrictor and stimulates aldosterone secretion. This causes hypertension. Several commercially-available drugs, such as losartan (2-butyl-4-chloro-1-[p-(o-1H-tetrazol-5-ylphenyl)benzyl]imidazole-5-methanol monopotassium salt), valsartan (N-(1-oxopentyl)-N-[[2′-(1H-tetrazol-5-yl) [1,1′-biphenyl]-4-yl]methyl]-L-valine), candesartan ((±)-1-[[(cyclohexyloxy)carbonyl] oxy]ethyl 2-ethoxy-1-[[2′-(1H-tetrazol-5-yl)[1,1′-biphenyl]-4-yl]methyl]-1H-benzimidazole-7-carboxylate), and telmisartan (4′-[(1,4′-dimethyl-2′-propyl [2,6′-bi-1H-benzimidazol]-1′-yl)methyl]-[1,1′-biphenyl]-2-carboxylic acid.) block Ang I and Ang II receptors and are effective to treat hypertension.

Circulating immune cells are known to play a significant role in nearly all forms of progressive kidney disease. Thus, the clinical success of kidney transplantation as a treatment for renal failure is highly sensitive to immune cell response. Circulating immune cells can wreak havoc in an allograft by triggering acute rejection episodes or by setting the stage for chronic immunoreactivity and inflammation. These responses are generally believed to lead to the development of Chronic Allograft Nephropathy (CAN), a pathologic state associated with chronic inflammation and histopathologic changes of the transplanted kidney (1, 2).

Conventional immunosuppressive agents target gene regulation and other select aspects of peripheral blood mononuclear cell (PBMC) activity to exert their immunosuppressive effects. However, such agents cannot blanket every mechanism involved in immune cell activation. For example, experimental evidence shows that Ang II also affects PBMCs in a manner akin to that of cytokines, stimulating the production of tumor necrosis factor (TNF-α), transforming growth factor (TGF-β), monocyte chemotactic protein (MCP-l), and interleukins (IL-1β) (3). Ang II also stimulates the production of tissue factor in PBMCs (4) and initiates a variety of signaling pathways that potentially transactivate other cytokine pathways, including the JAK-STAT pathway (5-10). Further, Ang II affects dendritic cell differentiation and, in vitro, leads to lymphocyte proliferation (11, 12).

The cellular effects of Ang II are mediated via cell surface receptors. The vast majority of the known physiologic effects of Ang II are mediated by Ang II binding to type I Ang II receptors (AT₁R). AT₁R receptors are present on T lymphocytes and monocyte/macrophages (12-14).

Because T lymphocytes and monocyte/macrophages play a central role in inflammation, a need exists for a method of manipulating AT₁R receptors so as to achieve a beneficial effect on the success of organ transplantation, especially kidney transplantation, and treatments of CAN.

### SUMMARY OF THE INVENTION

A first embodiment of the invention is a method of inhibiting production of IFN-Γ in patients having a transplanted organ. The method comprises administering to the patient having a transplanted organ an amount of an angiotensin receptor-blocking (ARB) compound, the amount being effective to inhibit production of IFN-Γ by T cells. In a preferred embodiment, the method is used to treat patients undergoing kidney transplantation. It is preferred that an IFN-Γ-inhibiting amount of a type 1, angiotensin II receptor-blocking compound is administered to the patient. The preferred compounds for use are selected from the group consisting of losartan, valsartan, candesartan, and telmisartan.

A second embodiment of invention is a method of treating chronic allograft nephropathy (CAN). Here, the method comprises administering to a patient having, or suspected of having, chronic allograft nephropathy (CAN) an anti-CAN-effective amount of an angiotensin receptor-blocking compound. The amount administered to the patient is preferably effective to inhibit production of IFN-Γ by T cells.

Any ARB compound will be effective in the present invention. However, a preferred compound for use in the invention is a type I Ang II receptor-blocking compound including, but not limited to, losartan, valsartan, candesartan, and telmisartan.

As detailed herein, there is a paucity of data examining the effects of agents that block type I angiotensin II receptors (AT₁R) on purified T cells. The Examples included herein illustrate the effects of chronic AT₁R blockade on aspects of T cell activity in a group of kidney transplant recipients. The patients received either AT₁R blocking compounds or no treatment at all as part of their treatment regimen. Further, the Examples illustrate an in vitro model to determine the mechanism of AT₁R blockade in effector T cells.

The Examples also demonstrate that AT₁R blockade in effector T cells results in a reduction in IFN-Γ generation. Reducing the IFN-Γ is thought to be due to a reduction in the number of activated macrophages. Thus, ARBs acting via this mechanism can offer protection from inflammation by preventing macrophage activation and release of toxic molecules.

The Examples further show that ARBs down-modulate T cell effector responses. These effects are demonstrated in vivo in the context of chronic allograft nephropathy (CAN). Fifteen individuals with biopsy-proven early CAN and high blood pressure were randomized to two groups: those receiving losartan, an ARB (n=8) or no ARB treatment (control; n=7) (as part of their overall treatment regimen). Urine samples showed a significant decrease in urinary H₂O₂ excretion in the ARB cohort. This indicates a reduction in intragraft inflammation. Peripheral blood lymphocytes (PBLs) were assayed for proliferation and interferon-gamma (IFN-Γ) generation. The ARB cohort had a significant reduction in T lymphocyte proliferation indices versus the control cohort and a decrease in T cell IFN-Γ production (as assessed by ELISpot). In vitro analysis of purified, stimulated peripheral blood T cells and an effector cytotoxic T lymphocyte (CTL) line both showed Ang II binding specific for AT₁R. The CTL line was used to demonstrate that blocking of AT₁R signaling with candesartan significantly reduced IFN-Γ expression as determined by intracellular staining.

Thus, blockade of AT₁R signaling with ARBs is shown to be useful and beneficial as a treatment for CAN and possibly in other T cell-mediated inflammatory disorders, in part, due to its suppressive effect on T cell growth and effector mechanisms.

### BRIEF DESCRIPTION OF THE DRAWINGS

FIG. 1A is a graph depicting the systolic blood pressure in the study cohort. (The study cohort is described in the Examples.) Blood pressure was assessed at every clinic visit at 3-month intervals. There were no significant differences in systolic BP between the ARB-treated (-o-) versus non-ARB-treated (-▪-) cohorts. Data shown are mean±standard deviation.”

FIG. 1B is a graph depicting the estimated glomerular filtration rate (eGFR) in the study cohort. Estimated GFR was calculated using the second equation derived by Nankivell et al. at the time points designated in the Examples. There were no significant differences in eGFR between the two study cohorts at each time point. The eGFR in the ARB-treated cohort (-o-) declined slightly after treatment as compared to the control group (-▪-). Data shown are mean±standard deviation.

FIG. 2 is a histogram depicting lymphocyte proliferation indices. Peripheral blood lymphocyte (PBL) samples were harvested from individuals in each study cohort and treated as described in the Examples to determine whether chronic ARB treatment altered lymphocyte proliferation. The lymphocyte proliferation index was calculated as described in the Examples. Chronic ARB treatment led to a decrease in PBL proliferation at six (p<0.005 vs. non-ARB treated cohort (G)) and nine (p<0.05 vs. non-ARB treated cohort (G)). Data shown are mean±standard deviation.

FIG. 3 is a histogram depicting Δ proliferation indices. The Δ Proliferation index was calculated as described in the Examples. This was done to determine if there was a change from baseline for each individual. Data shown are mean±standard deviation for individuals in each cohort. At each time point, the ARB-treated individuals demonstrated a significant reduction in PBL proliferation as compared to baseline (p<0.04 for each vs. non-ARB-treated individuals (G)).

FIG. 4 is a histogram depicting ELISpot results. This histogram demonstrates the ELISpot results for IFN-Γ comparing the non-ARB-treatment group (G) versus the ARB cohort. ELISpot assays were performed as described in the Examples. Values shown are mean±standard deviation of spots per 1×10⁶ cells. The ARB cohort had significantly fewer spots at six months (p=0.05 vs. non-ARB-treatment group) and nine months (p<0.001 vs. non-ARB-treatment group).

FIG. 5 is a histogram depicting [³H]-Ang II binding to AT₁R on human T cells. Primary human T cells were isolated and purified using ADYNABEAD™-brand magnetic particles (Dynal Biotech) coated with anti-CD4 and anti-CDS antibodies before being analyzed for the presence of AT₁R. Resting cells were purified immediately after isolation and proliferating cells were purified after 3 days of stimulation with an anti-CD3 antibody and rhIL-2. Effector cell binding was analyzed after 7 days of antigen-specific stimulation of a human CTL line which recognizes the p53 264-272 peptide. AT₁R binding is defined as the number of counts specifically blocked by the ARB candesartan and is determined by subtracting the counts bound in the presence of candesartan from the total binding. These findings are representative of three experiments; see Examples.

FIG. 6 is a flow cytometric graph showing that candesartan blocks IFN-Γ production in a human CTL line. Intracellular cytokine staining and flow cytometric analysis of a human CTL line specific for the p53 264-272 peptide was carried out after 4 days of antigen-specific stimulation in the presence or absence of the ARB candesartan (10 μM). Unstimulated cells received neither peptide nor candesartan during the growth period. Cells were fixed, permeabilized, and stained with antibodies specific for human IFN-Γ (labeled with phycoerythrin, PE) and IL-4 (labeled with fluorescein isothyiocyanate, FITC). These findings are representative of three experiments.

### DETAILED DESCRIPTION OF THE INVENTION

Abbreviations and Definitions:

The following abbreviations and definitions are used throughout the specification and claims. Those terms not explicitly defined herein take the standard and accepted meanings in the fields of medicine, chemistry, biochemistry, pathology, and/or immunology, as context dictates.
ACE-I=angiotensin-converting enzyme inhibitor.
Ang II=angiotensin II.
ARB=angiotensin receptor blocker.
AT₁R=type 1 angiotensin II receptor.
AT₂R=type 2 angiotensin II receptor.
BSA=bovine serum albumin.
CAN=chronic allograft nephropathy.
CNI=calcineurin inhibitor.
CsA=cyclosporine A.
CTL=cytotoxic T lymphocytes.
eGFR=estimated glomerular filtration rate.

ELISpot=Enzyme-linked ImmunoSpot. The ELISpot assay is a simple and highly sensitive assay for analyzing cell activation at the single-cell level. It is particularly useful for analyzing specific immune responses to whole antigens or peptides. Depending on the cytokine analyzed, the assay can be used to identify and discriminate between responses by different subsets of T cells (e.g., Th1 and Th2 cells). Reagents and hardware to perform ELISpot assays are available from numerous commercial sources, including Cellular Technology, Ltd. (d/b/a CTL, Cleveland, Ohio), Mabtech Inc., USA (Mariemont, Ohio) and eBioscience (San Diego, Calif.).
FCS=fetal calf serum.
FITC=fluorescein isothyiocyanate.
IFN-Γ=interferon-gamma.
MMF=mycophenolate mofetil.
N.S.=not significant.
PBL=peripheral blood lymphocyte.
PE=phycoerythrin.
PBMC=peripheral blood mononuclear cell.
PBS=phosphate-buffered saline.
SBP=systolic blood pressure.
Scr=serum creatinine.
STAT=signal transducers and activators of transcription.
TNF-α=tumor necrosis factor alpha.
TGF-β=transforming growth factor beta.
   Introduction:

A variety of in vitro studies have suggested that Ang II acts as an immunomodulatory peptide. However, these studies have never been extended to in vivo application, nor have AT₁R blockers been used to treat chronic allograft nephropathy. Thus, a straightforward, randomized, controlled study was performed (see Examples) to assess whether blockade of Ang II binding to the AT₁R affects T cell response in vivo. As shown in the Examples, chronic ARB administration in the setting of biopsy-proven CAN altered inflammatory status (as shown by urinary H₂O₂ excretion levels), PBL proliferative properties, and IFN-Γ generation.

Especially noteworthy is the finding indicating that in vivo immunomodulatory activity can be achieved by chronic ARB use. Further, the in vivo studies presented in the Examples, supported by the in vitro findings presented in the Examples, have identified angiotensin receptor signaling as a regulator of IFN-Γ expression in T cells.

Angiotensin receptor signaling is best studied in kidney transplant recipients because transplantation, by its very nature, involves T cell-mediated processes. Angiotensinogen and angiotensin converting enzyme (ACE) mRNA levels in allograft tissue are good indicators of function and outcome for kidneys transplanted in these patients (23).

All of the patients studied in the Examples had biopsy-proven CAN, a pathologic state characterized in part by persistent low-level inflammation (1, 2). Renin-angiotensin also has a direct role in potentiating CAN, as demonstrated in an animal model of CAN (16).

The Examples show that blocking Ang II signaling has beneficial effects on conditions characterized by inappropriate T cell activation. Such an approach is highly useful in treating CAN.

Further, the Examples show that stimulated effector T cells (represented by an antigen-specific human cytotoxic T lymphocyte cell line), have [³H]-angiotensin II binding above and beyond that seen in the stimulated proliferating T cells isolated from donors. Further, CD4+/CD8+ ratios differed in resting, proliferating, and effector populations (2:1, 1:2, 1:2.5, respectively). Because effector T cells demonstrate high levels of specific Ang II binding compared to primary peripheral T cells, either resting or stimulated, the AT₁R signaling pathway may play a role in regulating effector T cell functions.

Due to the high-level expression of AT₁R on the cell surface of T cells committed to the effector phenotype illustrated by the Examples, AT₁R is a candidate as a marker for activated T cells. The increased level of AT₁R on proliferating versus resting T cells also suggests that AT₁R signaling may be an important T cell activation signal. Further, the higher level of AT₁R expressed on effector I cells implies that committed cells may be more responsive to signaling through AT₁R.

The importance of AT₁R in stimulating lymphocyte proliferation in vivo is well known (14). Aug II, acting through AT₁R, has been shown to stimulate lymphocyte proliferation and required the activation of calcineurin phosphatase. The Examples, however, show that ARBs may be used in conjunction with calcineurin inhibitors with an additive immunomodulatory effect. For instance, the Examples show no significant differences in calcineurin inhibitor use between the ARB and control cohorts.

The implications of ARB treatment affecting PBL IFN-Γ generation are widespread. Aug II stimulates a three-to-five fold increase in lymphocyte IFN-Γ production in a biphasic manner (29). As shown in the Examples, this effect can be blocked in vitro by using a competitive AT₁R antagonist. Rather than measure systemic IFN-Γ concentrations, the Examples focus specifically on IFN-Γ generation by T cells from PBLs. The ARB treatment specifically decreases IFN-Γ production and was not accompanied by an increase Th2 type (IL-4) cytokine. This suggests a direct Ang II effect on IFN-Γ generation by Th1 CD4⁺ and CD8⁺ T cells.

Further, the in vitro model presented in the Examples confirms the in vivo observation regarding IFN-Γ inhibition using a different ARB. It should be noted that there are no significant differences among the two ARBs used in this study (losartan and candesartan). Agents such as losartan and candesartan that bind AT₁R also block AT₁R signal transduction. This can have significant intracellular effects as AT₁R initiate a number of different signaling pathways, including the Jak/STAT pathway (8, 10).

STAT1 (30) and STAT3 (31) have also been shown to be directly involved in IFN-Γ signaling. Interestingly, losartan treatment has been shown to decrease phosphorylation of both STATs (32, 33), possibly via activation of AT₂R signaling. A reduction in STATI phosphorylation could also result in inhibition of the transcription factor, T-bet.

The T-bet transcription factor plays a key role in stimulating IFN-Γ production (34), such as in a disease state such as CAN, where an excess of Aug II is produced (16) in the presence of chronic AT₁R inhibition. Alternatively, it is possible that either chronic AT₁R blockade or Aug II signaling through AT₂R actually increases suppression of cytokine signaling proteins (SOCS).

SOCS-1, SOCS-3, and the cytokine-inducible SH₂-containing protein (CIS) all play important roles in regulating cytokines, among them IFN-Γ (35, 36). Inhibition of AT₁R-dependent NF-AT activation (24) may also lead to inhibition of IFN-Γ production.

AT₁R blockade also resulted in a significant antioxidant effect, evidenced by the reduction in urinary H₂O₂ excretion in the ARB-treated cohort. ARBs, in general, abrogate oxidative stress (37, 38), in part, by inhibiting the production of ROS by macrophages through decreased transcription of NADH oxidase (39, 40). The interplay between T cells, IFN-g, and oxidative stress likely results from a series of events similar to those proposed by Libby and Pober (45). The immune modulation model they proposed outlines a cycle of events leading to chronic rejection. The cycle is initiated by some form of injury that activates the innate immune system and directs a Thi type response to the allogratt. This response leads to T cell infiltration, followed by IFN-Γ production, macrophage activation, and the release of ROS.

The production of IFN-Γ not only stimulates macrophages, but also acts on neighboring CD4+ T cells to promote differentiation to Thi cells. Further, IFN-Γ promotes pathological vascular remodeling. These are all factors commonly found in chronic allograft injury. For instance, increased frequencies of CD4+ T cells reactive against donor HLA peptides have been reported in CAN patients (41). Further, T cell clones isolated from individuals with chronic rejection consistently produced IL-2 and IFN-Γ while those from individuals with stable graft function did not (42). Models of chronic allograft injury also place CD4+, CD8+ T cells and macrophages in the allograft tissue (43). Finally, increased interstitial ROS are present in patients with chronic renal transplant failure (44). Taken together, these findings establish the presence of both IFN-Γ producing T cells and macrophages at the site of graft rejection. Thus, ARBs could have a markedly beneficial effect on both circulating lymphocytes and intragraft oxidative stress. By breaking the chronic inflammatory cycle at one of two points either through inhibition of production of IFN-Γ by T cells or by directly acting on macrophages to down-regulate the production of ROS, ARBs can help to decrease inflammation in transplant sites.

The physiological effects shown in T cells derived from PBL of CAN patients are not the only possible beneficial effects of ARB treatment. For instance, ARB treatment can decrease PBL DNA damage, potentially by altering PBL proliferative properties (46). At the same time, ARBs may have beneficial effects on other immune cells, decreasing tissue factor generation in monocyte/macrophages (4) and inhibiting dendritic cell maturation (11). Thus, ARBs have a wide range of beneficial immunomodulatory effects on mononuclear cells.

These conclusions are supported by the Examples, even having a small number of patients. Serial sampling and the ability to use subjects as their own controls overcome any statistical concerns.

In summary, ARBs have an immunomodulatory effect in vivo which is, in part, mediated through changes in IFN-Γ production. Concurrent studies in a CTL line corroborated the in vivo findings as ARB treatment decreased CTL IFN-Γ generation without a Th1-TH2 shift. Therefore, the immune effects of Ang II are significant and ARBs may have clinically televant immunomodulatory functions as a component of their receptor antagonism.

Preferred Compounds for Use in the Invention:

The present invention will function with any type of type 1, angiotensin II receptor (AT₁R) blocker. The preferred compounds for use in the invention, however, are losartan, valsartan, candesartan, and telmisartan. This list is by way of illustration and does not limit the scope of the invention disclosed and claimed herein in any fashion. All are AT₁R blockers and all have been approved by the FDA for treating hypertension.

Losartan, which is generally presented as its potassium salt, is a non-peptide molecule bearing the systematic name 2-butyl-4-chloro-1[p-(-o-1H-tetrazol-5-ylphenyl)benzyl]imidazole-5-methanol monopotassium salt. Losartan potassium is a white to off-white, free-flowing crystalline powder with a molecular weight of 461.01. It is freely soluble in water, soluble in alcohols, and slightly soluble in common organic solvents, such as acetonitrile and methyl ethyl ketone. Oxidation of the 5-hydroxymethyl group on the imidazole ring results in the active metabolite of losartan. It is available commercially under the trademark ACOZAAR marketed by Merck & Co., Inc., Whitehouse Station, N.J. ACOZAAR-brand losartan can be obtained commercially for oral administration containing either 25 mg or 50 mg of losartan potassium and the following inactive ingredients: microcrystalline cellulose, lactose hydrous, pre-gelatinized starch, magnesium stearate, hydroxypropyl cellulose, hydroxypropyl methylcellulose, titanium dioxide, D&C yellow No. 10 aluminum lake, and FD&C blue No. 2 aluminum lake. Both losartan and its pharmaceutically-active metabolite are highly bound to plasma proteins in vivo (plasma-free fractions are 1.3% and 0.2%, respectively). Losartan's FDA-approved labeling indicates that it is to be used to treat hypertension.

Valsartan is a valine-based compound having the systematic designation N-(1-oxopentyl)-N-[(2′-(1H-tetrazol-5-yl)[1,1′-biphenyl]-4-yl]methyl]-L-valine. Valsartan is a white to practically white fine powder. It is soluble in ethanol and methanol and slightly soluble in water. It is available commercially under the trademark DIOVAN. DIOVAN-brand valsartan is available as capsules for oral administration, containing either 80 mg or 160 mg of valsartan. The inactive ingredients of the capsules are cellulose compounds, crospovidone, gelatin, iron oxides, magnesium stearate, povidone, sodium lauryl sulfate, and titanium dioxide. Valsartan is commercially marketed by Novartis Pharmaceuticals Corporation, East Hanover, N.J.

Candesartan cilexetil (referred to herein simply as candesartan) is a nonpeptide compound having the systematic name (±)-1-[[(cyclohexyloxy)carbonyl]oxy]ethyl 2-ethoxy-1-[[2′-(1H-tetrazol-5-yl)[1,1′-biphenyl]-4-yl]methyl]-1H-benzimidazole-7-carboxylate. Candesartan is a white to off-white powder with a molecular weight of 610.67. It is practically insoluble in water and sparingly soluble in methanol. Candesartan is a racemic mixture containing one chiral center at the cyclohexyloxycarbonyloxy ethyl ester group. Following oral administration, candesartan undergoes hydrolysis at the ester link to form the active drug (candesartan proper) which is achiral. Candesartan is marketed under the ATACAND trademark by AstraZeneca Pharmaceuticals LP, Wilmington, Del. ATACAND-brand candesartan is available commercially for oral use (to treat hypertension) as tablets containing either 4 mg, 8 mg, 16 mg or 32 mg of candesartan cilexetil. The commercially available formulation also includes the following inactive ingredients: hydroxypropyl cellulose, polyethylene glycol, lactose, corn starch, carboxymethylcellulose calcium, and magnesium stearate. Ferric oxide reddish brown is added to the 8 mg, 16 mg, and 32 mg tablets as a colorant.

Telmisartan is a nonpeptide compound having the systematic name 4′-[(1,4′-dimethyl-2′-propyl [2,6′-bi-1H-benzimidazol]-1′-yl)methyl]-[1,1′-biphenyl]-2-carboxylic acid. Telmisartan is a white to slightly yellowish solid. It is practically insoluble in water within a pH range of from 3 to 9, is only sparingly soluble in strong acids (except HCl), and is soluble in strong bases. Telmisartan is marketed commercially under the trademark MICARDIS by Boehringer Ingelheim Pharmaceuticals, Inc., Ridgefield, Conn. MICARDIS-brand telmisartan is available as oral tablets (for treating hypertension) in dosages containing 20, 40, or 80 mg of telmisartan. The commercial tablets contain the following inactive ingredients: sodium hydroxide, meglumine, povidone, sorbitol, and magnesium stearate. The tablets are hygroscopic and must be protected from moisture during storage.

### EXAMPLES

The following Examples are included solely to provide a more complete and thorough illustration of the invention disclosed and claimed herein. The Examples do not limit the scope of the invention claimed herein in any fashion.

### Example 1

### The Study Population

The study, enrollment process, and protocols described herein were approved by the University of Wisconsin Institutional Review Board. Individuals with evidence of chronic allograft dysfunction have allograft functionality greater than 6 months with serum creatinine≧0.3 mg/dl above discharge creatinine. In the absence of acute rejection, these individuals have obstructive uropathy or urinary tract infection. To be eligible for the study, individuals also had concomitant evidence of elevated blood pressure (>140/80) and/or proteinuria (>2+ on dipstick urinalysis or spot urine protein-to-creatinine ratio>1).

Once identified, study subjects were randomized and treated with either losartan (25-100 mg) as part of their anti-hypertensive regimen or with other types of anti-hypertensive agents, e.g., calcium channel blockers. Agents functioning as AT₁R blockers were excluded. Neither group received ACE inhibitors. Study subjects were maintained on their standard immunosuppressive regimen (corticosteroids 5-10 mg orally daily; mycophenolate mofetil 500 to 1000 mg orally twice a day; and calcineurin inhibition with either tacrolimus (target levels 2-5 ng/ml), cyclosporine A (target levels 100-150 ng/ml), or rapamycin (target levels 5-15 ng/ml ).

Study subjects underwent biopsies at the initiation of the study. Simultaneously, whole blood samples were obtained for analysis of peripheral blood lymphocyte proliferation and stimulated cytokine production. Subsequent whole blood samples were obtained at three-month intervals for serial analyses. Blood samples and serial assessment of transplant function, blood pressure, and proteinuria were performed in conjunction with the University of Wisconsin General Clinical Research Center (GCRC). Estimated glomerular filtration rate (eGFR) was assessed using the second equation derived by Nankivell. (See Transplant Proc. August (2003), 35(5):1671-2.)

### Example 2

### Detection of Urinary Hydrogen Peroxide (H₂O₂)

The Amplex Red Hydrogen Peroxide/Peroxidase Assay Kit (Molecular Probes, Eugene, Ore.) was used according to the manufacturer's instructions to measure H₂O₂. Briefly, urine samples were centrifuged at 1000 rpm for 10 minutes at room temperature. Serial 50 μl dilutions of H₂O₂ (standard curve), and urine supernatants were placed into individual wells of a 96-well microplate. A 50 μl volume of the Amplex Red reagent/HRP solution was then added to each microplate well containing the standards and samples. The plate was incubated at room temperature for 30 minutes, protected from light. A microplate reader with fluorescence emission detection at 590 nm was used to measure H₂O₂ levels. H₂O₂ concentrations were read from the standard curve.

### Example 3

### Peripheral Blood Lymphocyte (PBL) Harvest From CAN Patients

Eight to nine milliliters of whole blood taken at each time point from each individual were incubated at room temperature in 40 ml ACK lysis buffer (150 mM NH₄Cl, 10 mM KHCO₃, 0.1 mM Na₂EDTA, pH 7.3) for 10 minutes to lyse red blood cells. Samples were then centrifuged at 1000 rpm. The supernatant was discarded. Cells were resuspended in 10 ml complete RPMI media (RPMI-1640, BioWhitaker, Walkersville, Md.) supplemented with FCS (Sigma Aldrich, St. Louis, Mo.); 2 mM L-glutamine (BioWhitaker); 50 μM 2-β-mercaptoethanol (Sigma Aldrich); 100 U/ml penicillin; and 100 μg/ml streptomycin sulfate (Sigma Aldrich).

Twenty μl of this sample was removed for cell enumeration. Forty ml of complete RPMI media was then added to the remaining sample and this was centrifuged at 1000 rpm for 10 minutes. The resulting PBL pellet was resuspended in complete RPMI media at a concentration of 1.5×10⁶ cells per ml.

### Example 4

### Generation of CD3/CD28-Coated Plates

CD3/CD28-coated plates were coated with antibody diluted in PBS (1 mg/mI). Anti-CD3ε mAb (Immunotech, Marseille, France) and anti-human CD28 mAb (PharMingen, a wholly-owned subsidiary of BD Biosciences, San Diego, Calif.) were used to stimulate T cells in vitro. Control wells were exposed to plain PBS. These plates were incubated at 4EC overnight before use.

### Example 5

### Proliferation Assay

Plates prepared as above were rinsed twice with PBS prior to adding cells. Cells were plated at 1×10⁶ cells per well. Cells were then incubated in the six-well plates for three days at 37EC. Cells exposed to CD3/CD28-coated wells or control wells were harvested into 15 ml conical tubes and centrifuged at 1000 rpm for 10 minutes. The remaining cell pellet was suspended in complete RPMI media at a concentration of 1×10⁶ cells per ml. Control and CD3/CD28-treated cells were plated at 1×10⁵ cells per well in triplicate in a flat bottom plate. Media (100 μl) containing 0.5 μCu [³H]-thymidine (Amersham Biosciences, Piscataway, N.J.) was added to each well and the plates were incubated overnight at 37EC. Some plates were analyzed immediately after the overnight incubation. Other plates were frozen at 20EC and then analyzed within 30 days of the initial study. [³H]-Thymidine incorporation was then determined using a Packard TopCount NXT scintillation plate reader. The proliferation index was calculated as previously described (18). The Δ proliferation index was then calculated by assessing the difference between the proliferation index at baseline and the proliferation index at various study time points as indicated.

### Example 6

### ELISpot Assays

ELISpot assays were conducted according to the manufacturer's directions (Cellular Technology Ltd., Cleveland, Ohio). Briefly, after plating cells in the proliferation assay, the balance of the cells unused in that assay were used for the ELISpot assay at a concentration of 3×10⁶ cells per ml. One hundred μl of RPMI media were added to each well with a multichannel pipetter. Aliquots of treated and untreated cells (300 μl) were added to the IFN-Γ and IL-5 wells with serial two-fold dilutions. The last row served as a media control.

ELISpot plates were incubated for two days at 37EC. Plates were washed three times with PBS and then washed three times with PBS/Tween. Secondary detection antibodies (OptElA, IFN, and IL-5 obtained from the aforementioned kits) were diluted at 4 μl/ml in 1% BSA/PBS and added to the plates for 24 hours at 4EC. The plates were then washed four times with PBS/Tween. Streptavidin alkaline phosphatase 1:1000 (Dako Cytomation, Glostrop, Denmark, catalog no. D0396) 100 μl/well diluted in 1% BSA/PBS/Tween was then added to the plates at room temperature for two hours. The plates were again washed four times with PBS and 200 μl of substrate was added to each well. Substrate was obtained by mixing 66 μl of 0.25 g NBT in 70% DMF/30% H₂O; 0.25 g BCIP (Sigma) in 5 mL 100% DMF with 10 ml NBT buffer [12.44 g Tris; 5.89 g NaCl, 1.0 g MgCl₂ (hexa hydrate); pH 9.5]. Plate development occurred over a 15-60 minute time frame (depending on the cytokine and the patient). At full development, plates were washed with distilled water to stop the reaction and allowed to air dry. Plate images were then scanned into computer images and spots analyzed using Immunospot-brand software (from CTL Technologies).

### Example 7

### Isolation of Purified Human T Cells for Binding Assays

Blood was collected from volunteer donors following prior approval by the Institutional Review Board at Texas Tech University Health Sciences Center. Initially, lymphocytes were enriched using a Ficoll-paque density gradient. Cells were removed, washed in PBS, resuspended in complete RPMI media, counted, transferred to 6-well plates at 1×10⁶ cells/well, and incubated for 6 h at 37EC to remove adherent cells (i.e., monocytes and macrophages). Non-adherent cells (lymphocytes) were collected and used in the experiments as follows: resting T cells were prepared from the non-adherent cell sample using DYNABEAD-brand magnetic particles (Dynal Biotech; Oslo, Norway, and Lake Success, New York, USA) coated with anti-CD4 and antiCD8 antibodies, following the manufacturer's instructions. After washing the beads to remove unbound cells, the T cells were eluted from the beads and incubated at 37EC in complete RPMI media. Cells were stained to evaluate the purity of the T cells using a cocktail of antibodies (anti-CD3-PE/CD4-FITC-tagged antibodies or anti-CD3-PE/CD8-FITC-tagged antibodies). Anti-CD3ε mAb (UCHT1, BD PharMingen, San Diego, Calif.) was used for flow cytometry. Anti-CD4 (RPA-T4) and CD8 (HIT8a) mAbs conjugated to either FITC or PE and isotype control antibody conjugates for mouse IgG₂ₐ,κ (G155-178) and mouse IgG_{2b},κ (27-35) were purchased from BD PharMingen.

### Example 8

### Stimulation of Primary T Cells for Ang II Binding Assays

Proliferating T cells were generated as follows: non-adherent cells (lymphocytes) were collected and stimulated with anti-CD3 (OKT3, 20 ng/ml) and recombinant human IL-2 (50 U/mL, R&D Systems, Minneapolis, Minn.). Three days later, DYNABEAD-brand magnetic beads coated with anti-CD4 and anti-CD8 antibodies were used to isolate proliferating CD4⁺ and CD8⁺ T cells. The isolated CD4⁺ and CD8⁺ T cells were then used in T cell binding assays at 10⁶-cells/binding reactions.

### Example 9

### Antigen-Specific Stimulation of the Anti-p53 CTL

A human effector T cell line specific for the human peptide derived from p53, a tumor-suppressor protein presented in the context of HLA-A2 was generated previously (19) and was kindly provided by Dr. Albert De Leo (University of Pittsburgh). This CTL line was used in binding assays and for characterizing the effects of AT₁R blockade on effector T cell expression of IFN-Γ. The effector T cell line was maintained using irradiated HLA-A2-matched feeder cells pulsed with the p53 peptide (amino acids 264-272: LLGRNSFEV) and supplemented with 10 U/ml of rhIL-2. Effector T cells were grown in six-well plates at 10⁶ cells/well and re-stimulated using an irradiated HLA-A2-positive lymphoblastoid cell line (LCL) at 1×10⁴/well pulsed with 100 μg of p53 peptide. Stimulated CTL were harvested 7 days later for Ang II binding studies.

### Example 10

### Radio-Ligand Binding Studies Using [³H]Angiotensin II

Receptor binding assays were performed as previously described (20). Briefly, isolated T cells were washed once in PBS and then resuspendend in ice-cold binding buffer containing 50 mM Tris HCL (pH 7.5), 125 mM NaCl, 4 mM KCl, 5 mM MgCl₂, 1 mM CaCl₂, 10 μg/ml Bacitracin, 2 mg/ml Dextrose, and 2.5 mg/ml BSA. Aliquots of 10⁶ T cells (resting (unstimulated), proliferating (stimulated), and effector) were incubated in triplicate with 15 nM [³H]angiotensin II for 30 minutes at room temperature with or without excess concentrations of candesartan (10 μM) that was kindly provided by AstraZeneca (Wilmington, Del.). The incubation mixtures were filtered through a 96-well silent screen plate (Loprodyne membrane, 0.45 μm pore; Nagle Nunc International, Rochester, N.Y.). After washing, cell-bound labeled counts were determined in a beta scintillation counter, and AT₁R-specific binding was calculated using standard formulas (19). Resting T cells were prepared as described above and used directly in binding assays. Stimulated primary and effector T cells were prepared as explained above before use in the binding assay.

### Example 11

### Intracellular Staining

The human CTL line was added to a 96-well plate (2×10⁵/well) and stimulated using an irradiated lymphoblastoid cell line (LCL) at 5×10⁴/well pulsed with 100 μg of p53 peptide. Cells were cultured in the absence (positive control) or presence of 10 μM candesartan. At 96 hours, the cells were harvested and intracellular cytokine staining was performed according to standard protocol (21). Intracellular staining of the human CTL controls (untreated/unstimulated and untreated/stimulated) and the experimental group (candesartan treated/stimulated) was performed using antibodies specific for cytokines IL-4 (anti-IL-4, FITC-conjugated, mouse IgG_{2b},κ) and IFN-Γ (anti-IFN-Γ, PE-conjugated, mouse IgG₂ₐ,κ), both from R&D Systems, catalog nos. 34019.111 and 25718.11, respectively, following the protocol provided by PharMingen.

In brief, stimulated effector T cells were harvested after 4 days of activation, washed in PBS supplemented with 1% BSA (Sigma-Aldrich). Cells (2-5×10⁵ cells/staining group) were incubated with purified unlabeled isotype control for 30 min. at room temperature. Cells were fixed using 2% paraformaldehyde, washed, chilled on ice and then resuspended in PBS containing 0.1% saponin and 1% BSA, and left on ice for 30 min. to permeabilize the membranes. The specific antibodies were added to the samples (isotype controls or anti-IL-4-FITC and LFN-Γ-PE mAbs) and incubated for an additional 30 minutes. The cells were washed 1× in PBS containing 1% BSA and then resuspended in PBS containing 0.5% paraformaldehyde and analyzed by flow cytometry.

### Example 12

### Flow Cytometry

Flow cytometric analysis for the detection of CD4+/CD8+ effector cells and for intracellular cytokine staining for IL-4 and IFN-Γ was performed using a FACScan-brand flow cytometer (BD BioSciences, San Jose, Calif.) running Cell Quest-brand software.

Statistical Methodology:

Differences between groups were assessed using Student's t-test and ANOVA. Statistical analyses were performed using SigmaStat-brand software for Windows, version 3.0 (SPSS Inc., Chicago, Ill.). Data are reported as mean±standard deviation.

### RESULTS

Study Population:

To examine the renoprotective effects of long-term ARB therapy in chronic allograft nephropathy (CAN), seventeen kidney transplant recipients with biopsy-proven CAN and high blood pressure were randomly divided into two groups. One group received losartan as part of their high blood pressure regimen; the other did not. Neither group received ACE inhibitors (see Table 1). There were more men than women in the control arm. Kidney function as assessed by mean serum creatinine and mean estimated glomerular filtration rate (eGFR) (22) were comparable between the two groups. A similar proportion of individuals in each group received calcineurin inhibitors during the time course of the study.

TABLE 1
Chronic Allograft Nephropathy Patient Demographics

| Category | Control | ARB | P value |
|---|---|---|---|
| N | 9 | 8 | |
| Gender | 5 M/4 F | 5 M/3 F | N.S. |
| Age (yrs) | 48.4 ± 14.2 | 53.6 ± 9.6 | N.S. |
| Mean Scr (mg/dl) | 1.82 ± 0.55 | 1.66 ± 0.3 | N.S. |
| CNI/Sirolimus | 6/3 | 4/1 | N.S. |
| History of Biopsy- | 2 | 2 | N.S. |
| Proven Acute | | | |
| Rejection | | | |
| Banff Score for | 6 Banff Grade I | 5 Banff Grade I | N.S. |
| Chronic/Sclerosing | | | |
| Allograft | 3 Banff Grade II | 3 Banff Grade II | |
| Nephropathy CADI | 4.1 ± 1.1 | 4.0 ± 1.4 | |
| Score | | | |
| Initial | All rec'd induction | All rec'd induction | |
| Immunosuppression | antibody therapy. | antibody therapy. | |
| | 7 rec'd CsA, | 7 rec'd CsA, | |
| | MMF, & | MMF, & | |
| | prednisone. | prednisone. | |
| | 2 rec'd tacrolimus, | 1 rec'd tacrolimus, | |
| | MMF, & | MMF, & | |
| | prednisone. | prednisone. | |

Scr: serum creatinine;
CNI: calcineurin inhibitor;
CsA: cyclosporine A;
MMF: mycophenolate mofetil

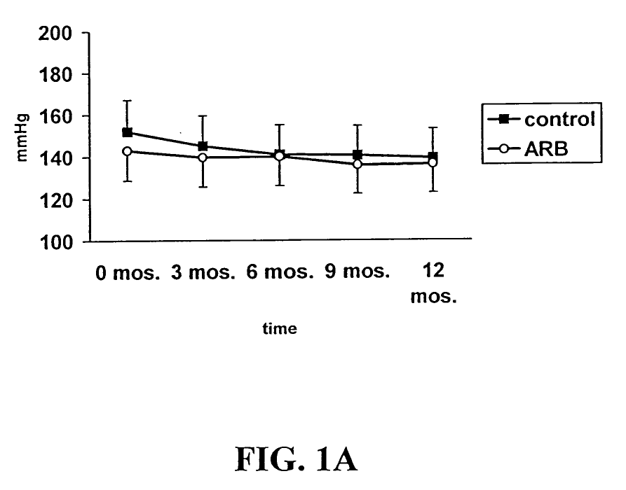

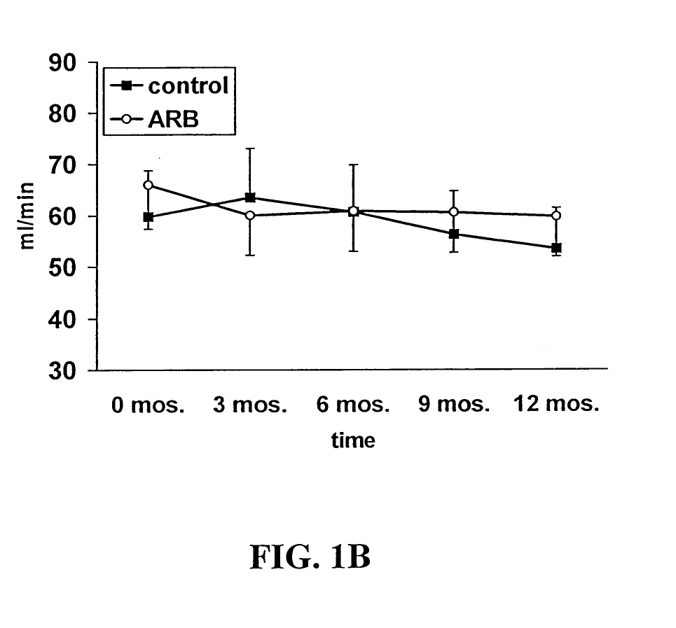

Blood pressure maintenance was similar in both groups, though slightly higher in the control population during the first three months (see FIG. 1A). Estimated GFR values were similar between the two groups, with the ARB cohort demonstrating a slight reduction in eGFR with institution of ARB therapy (FIG. 1B). These initial clinical observations suggest the existence of alternative mechanisms of action for losartan which are unrelated to blood pressure or eGFR effects.

Detection of Urinary Hydrogen Peroxide (H₂O₂):

Because inflammation can induce oxidative stress, urinary H₂O₂ was assayed as a surrogate marker of intragraft oxidative stress. Thus, urinary H₂O₂ levels are indicative of graft inflammation. There was no significant difference in baseline urinary H₂O₂ values between the ARB (2.51±0.62 μM) and control cohort (2.1±0.65 μM) (N.S.). The ARB cohort demonstrated a significant and stepwise reduction in urinary H₂O₂ excretion (6 months: 2.17±0.42 μM; 9 months: 1.65±0.49 μM; p<0.05 9 months vs. time 0), thus indicating that the treatment does reduce inflammation in the allograft. The control cohort did not demonstrate any significant reduction in urinary H₂O₂ excretion during the time course of the study.

These findings indicate that inhibition of AT₁R signaling has anti-inflammatory properties and that allograft inflammation in transplant patients can be reduced by administering AT₁R blocking compounds to the patient.

T Cell Proliferation Indices:

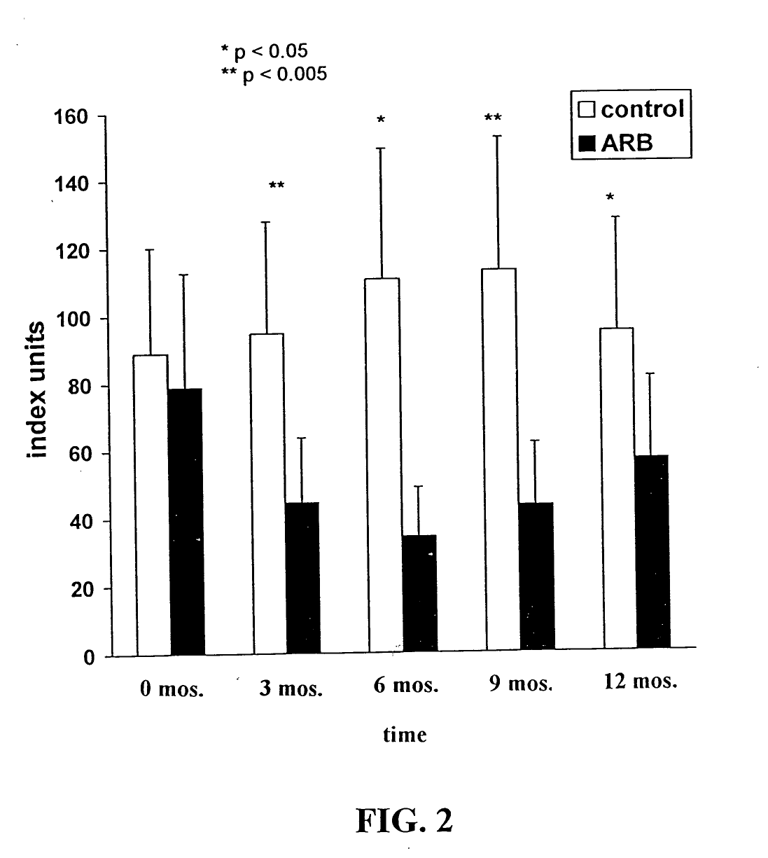

The reduction observed in urine H₂O₂ levels in losartan-treated patients implicates an anti-inflammatory mechanism as an explanation for the action of ARBs. Because T cells are critical in regulating (as well as causing) inflammation, the ability of ARBs to inhibit T cell proliferation was examined. To address this, PBLs and specifically stimulated T cells (CD4+ and CD8+) were isolated from the control patients (standard treatment) and patients treated with losartan and the standard treatment. Proliferation assays were then performed. The ARB cohort demonstrated a significant reduction in T lymphocyte proliferation indices after six and nine months of losartan treatment versus the control group (6 month 69±47% decrease; p<0.005; 9 month: 52±21% decrease; p<0.05) (see FIG. 2).

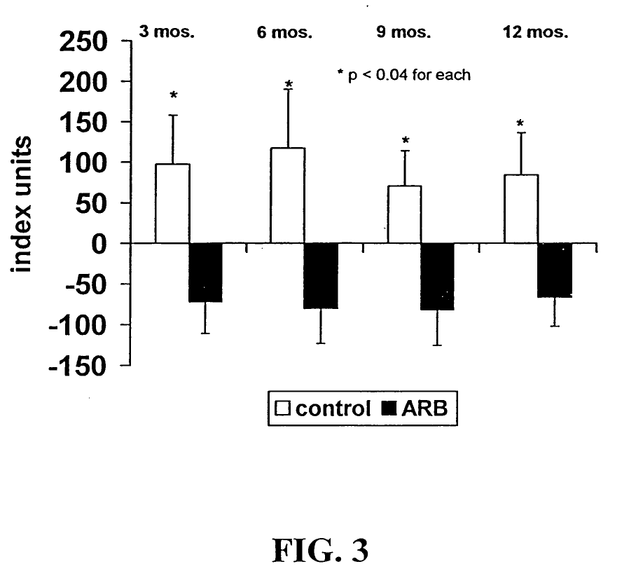

The change in proliferation from baseline was also analyzed (the Δ proliferation index). The ARB cohort had a significant reduction in the Δ proliferation index at each time point evaluated (p<0.04 at three, six, and nine months) (see FIG. 3). These findings suggest that losartan has immunomodulatory effects on T cell activity and that reducing T cell proliferation may be useful to prevent inflammation by suppressing the expansion of allogenic T cell clones. Thus, the present invention includes a method of suppressing expansion of allogenic T cell clones by administering compounds that block AT₁R receptors to a person in need thereof.

ELISpot Analyses for T Cell Generation of IFN-Γ:

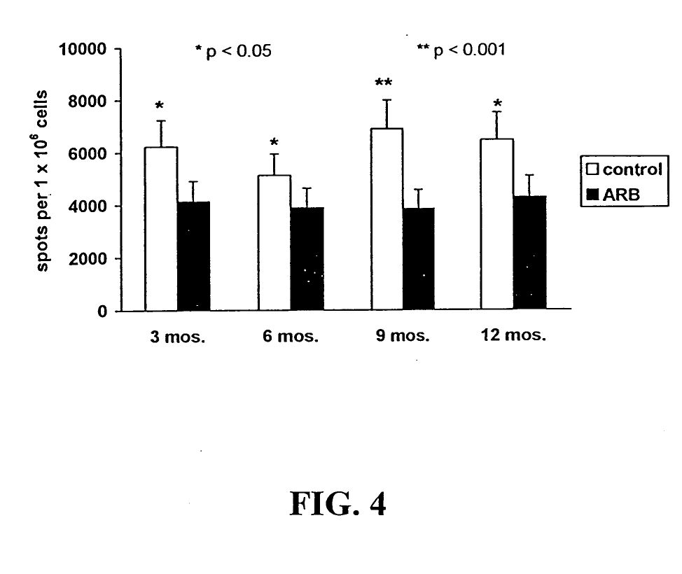

One of the key inflammatory mediators produced by T cells is the cytokine IFN-Γ. IFN-Γ, produced primarily by T cells and NK cells, has many effector functions, including, but not limited to, the activation of macrophages and endothelial cells. To examine whether ARBs affect IFN-Γ expression in T cells, cells were isolated from patients, stimulated in vitro, and assayed for cytokine expression. Baseline values were not significantly different between treatment groups (spots per 3×10⁵ cells study entry ARB: 3654±1471; control: 3803±1255; N.S.) The ARB cohort demonstrated a significant reduction in IFN-Γ producing cells (spots per 1×10⁶ cells; 3 months C ARB: 4127±1289; control: 6231±1834; p=0.01; 6 months C ARB: 3899±1508; control: 5124±2253; p=0.05, 9 months C ARB: 3845±1493; control: 6889±1863; p<0.001; 12 months C ARB: 4276±1456; control: 6471±1621; p<0.05) (see FIG. 4). There was no significant change in IL-4 or IL-5 production following institution of ARB therapy (data not shown). These findings show that AT₁R signaling is important for the up-regulation of effector T cell responses such as the production of the pro-inflammatory cytokine IFN-Γ. By reducing this effect, the present invention similarly reduced inflammation at the allograft.

Expression of AT₁R on Primary Human T Cells and Effector Cells:

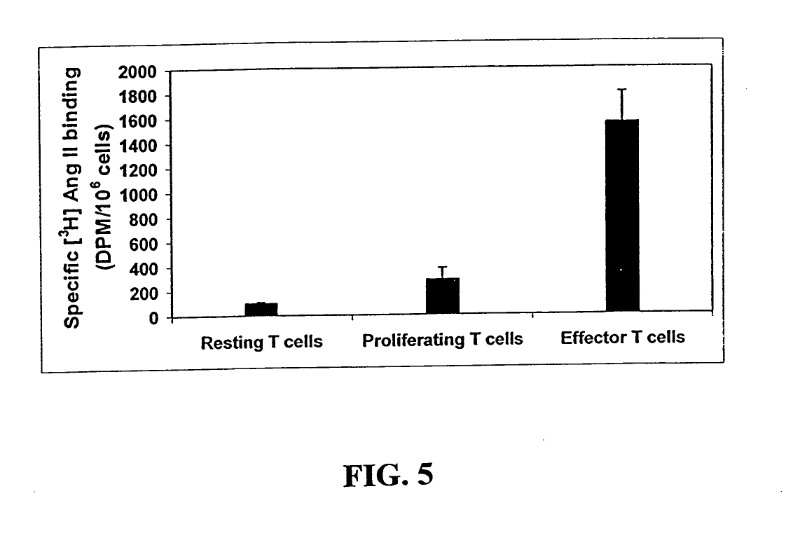

Although AT₁R expression has been described in murine T lymphocytes (14), the presence of AT₁R on human T lymphocytes remains unclear. Further, whether an AT₁R is constitutively expressed or differentially regulated in T lymphocytes remains unclear. Therefore, ligand binding studies were performed to determine the level of AT₁R expression in T cells (see FIG. 5). AT₁R were barely detectable on resting T cells. Proliferating T cells, however, demonstrated approximately a three-fold increase in binding as compared to resting T cells. This illustrates that stimulation increases AT₁R cell surface expression.

An in vitro model, consisting of a human CTL line specific for the 264 peptide from the human tumor suppressor protein p53, and presented in the context of human HLA-A2, was also used. Effector cells, generated by antigen-specific stimulation of this CTL line, displayed an approximately five-fold greater AT₁R surface expression over that seen in proliferating peripheral T cells.

In Vitro Effect of Ang II Blockade on IFN-Γ Production in a CTL Line:

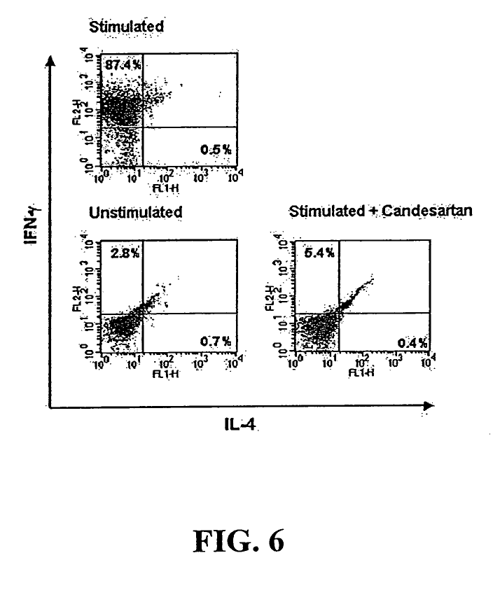

To elucidate the direct effects of an AT₁R antagonist on T cell functions, the CTL line was used for additional studies. Upon antigen-specific stimulation, this CTL line expresses detectable quantities of IFN-Γ. IFN-Γ was assayed by intracellular cytokine staining and flow cytometric analysis. Detection of IFN-Γ- and IL-4 cytokine-producing cells was carried out using anti-IFN-Γ-PE mAbs and anti-IL-4-FITC mAbs. Minimal non-specific staining of the CTL line was observed with isotype antibody controls. Unstimulated cells showed little production of either cytokine. After peptide antigen-specific stimulation, 87% of the T cells produced IFN-Γ. No change was noted in IL-4 production. Candesartan (10 μM treatment significantly reduced the number of IFN-Γ producing cells (>90% reduction) to a level seen in the unstimulated T cell population (see FIG. 6). A forward and side scatter profile for the cells evaluated (unstimulated, stimulated and treated/stimulated) were similar (not shown), indicating that the candesartan effect was specific.

These findings are consistent with the in vivo results, namely that ARBs inhibit T cell production of IFN-Γ. Further, these findings demonstrate that angiotensin receptor signaling in T cells is a key mechanism for regulating IFN-Γ expression.

### REFERENCES

Each of the following references is incorporated herein in its entirety.

1. Paul, L. C. 2001. Immunologic risk factors for chronic renal allograft dysfunction. Transplantation 71:SS17-23.

2. Paul, L. C. 2000. Chronic allograft nephropathy-a model of impaired repair from injury? Nephrol Dial Transplant 15:149-151.

3. Dorffel, Y., Latsch, C., Stuhlmuller, B., Schreiber, S., Scholze, S., Burmester, G. R., and Scholze, J. 1999. Preactivated peripheral blood monocytes in patients with essential hypertension. Hypertension 34:113-117.

4. Napoleone, E., Di Santo, A., Camera, M., Tremoli, F., and Lorenzet, R. 2000. Angiotensin-converting enzyme inhibitors down-regulate tissue factor synthesis in monocytes. Circ Res 86:139-143.

5. Chen, X., Wang, J., Zhou, F., Wang, X., and Feng, Z. 2003. STAT proteins mediate angiotensin IL-induced production of TIMP-I in human proximal tubular epithelial cells. Kidney Int 64:459-467.

6. Seebach, F. A., Welte, T., Fu, X. Y., Block, L. H., and Kashgarian, M. 2001. Differential activation of the STAT pathway by angiotensin II via angiotensin type 1 and type 2 receptors in cultured human fetal mesangial cells. Exp Mol Pathol 70:265-273.

7. Liang, H., Venema, V. J., Wang, X., Ju, H., Venema, R. C., and Marrero, M. B. 1999. Regulation of angiotensin II-induced phosphorylation of STAT3 in vascular smooth muscle cells. J Biol Chem 274:19846-19851.

8. Marrero, M. B., Venema, V. J., Ju, H., Eaton, D. C., and Venema, R. C. 1998. Regulation of angiotensin II-induced JAK2 tyrosine phosphorylation: roles of SHP-1 and SHP-2. Am J Physiol 275:C1216-1223.

9. Venema, R. C., Venema, V. J., Eaton, D. C., and Marrero, M. B. 1998. Angiotensin II-induced tyrosine phosphorylation of signal transducers and activators of transcription 1 is regulated by Janus-activated kinase 2 and Fyn kinases and mitogen-activated protein kinase phosphatase 1. J Biol Chem 273:30795-30800.

10. Marrero, M. B., Schieffer, B., Paxton, W. G., Heerdt, L., Berk, B. C., Delafontaine, P., and Bernstein, K. E. 1995. Direct stimulation of Jak/STAT pathway by the angiotensin II AT1 receptor. Nature 375:247-250.

11. Nahmod, K. A., Vermeulen, M. E., Raiden, S., Salamone, G., Gamberale, R., Fernandez-Calotti, P., Alvarez, A., Nahmod, V., Giordano, M., and Geffner, J. R. 2003. Control of dendritic cell differentiation by angiotensin II. FASEB J 17:491-493.

12. Kunert-Radek, J., Stepien, H., Komorowski, J., and Pawlikowski, M. 1994. Stimulatory effect of angiotensin II on the proliferation of mouse spleen lymphocytes in vitro is mediated via both types of angiotensin II receptors. Biochem Biophys Res Commun 198:1034-1039.

13. Shimada, K., and Yazaki, Y. 1978. Binding sites for angiotensin II in human mononuclear leucocytes. J Biochem (Tokyo) 84:1013-1015.

14. Nataraj, C., Oliverio, M. I., Mannon, R. B., Mannon, P. J., Audoly, L. P., Amuchastegui, C. S., Ruiz, P., Smithies, O., and Coffman, T. M. 1999. Angiotensin II regulates cellular immune responses through a calcineurin-dependent pathway. J Clin Invest 104:1693-1701.

15. Campistol, J. M., Inigo, P., Jimenez, W., Lario, S., Clesca, P. H., Oppenheimer, F., and Rivera, F. 1999. Losartan decreases plasma levels of TGF-betal in transplant patients with chronic allograft nephropathy. Kidney Int 56:714-719.

16. Ziai, F., Nagano, H., Kusaka, M., Coito, A. J., Troy, J. L., Nadeau, K. C., Rennke, H. G., Tilney, N. L., Brenner, B. M., and MacKenzie, H. S. 2000. Renal allograft protection with losartan in Fisher→Lewis rats: hemodynamics, macrophages, and cytokines. Kidney Int 57:2618-2625.

17. Ishikawa, A., and Kawabe, K. 2003. Beneficial effect of losartan against proteinuria from the renal allograft. Transplant Proc 35:289-290.

18. MacKenzie, D. A., Sollinger, H. W., and Hullett, D. A. 2000. Role of CD4+ regulatory T cells in hyperbaric oxygen-mediated immune nonresponsiveness. Hum Immunol 61:1320-1331.

19. Hoffmann, T. K., Loftus, D. J., Nakano, K., Maeurer, M. J., Chikamatsu, K., Appella, E., Whiteside, T. L., and DeLeo, A. B. 2002. The ability of variant peptides to reverse the nonresponsiveness of T lymphocytes to the wild-type sequence p53(264-272) epitope. J Immunol 168:1338-1347.

20. Thekkumkara, T. J., Thomas, W. G., Motel, T. J., and Baker, K. M. 1998. Functional role for the angiotensin II receptor (AT1A) 3′-untranslated region in determining cellular responses to agonist: evidence for recognition by RNA binding proteins. Biochem J 329 (Pt 2):255-264.

21. Coligan, J. E., Kruisbeek, A. M., Margulies, D. H., Shevach, E. M., and Strober, W., editors. 1994. Current Protocols in Immunology: Green Publishing Associates, Inc. and John Wiley & Sons.

22. Nankivell, B. J., Gruenewald, S. M., Allen, R. D., and Chapman, J. R. 1995. Predicting glomerular filtration rate after kidney transplantation. Transplantation 59:1683-1689.

23. Becker, B. N., Jacobson, L. M., Becker, Y. T., Radke, N. A., Heisey, D. M., Oberley, T. D., Pirsch, I D., Sollinger, H. W., Brazy, P. C., and Kirk, A. D. 2000. Renin-angiotensin system gene expression in post-transplant hypertension predicts allograft function. Transplantation 69:1485-1491.

24. Suzuki, Y., Gomez-Guerrero, C., Shirato, I., Lopez-Franco, O., Hemandez-Vargas, P., Sanjuan, G., Ruiz-Ortega, M., Sugaya, T., Okumura, K., Tomino, Y., et al. 2002. Susceptibility to T cell-mediated injury in immune complex disease is linked to local activation of renin-angiotensin system: the role of NF-AT pathway. J Immunol 169:4136-4146.

25. Tsutsumi, K., Stromberg, C., and Saavedra, J. M. 1992. Characterization of angiotensin II receptor subtypes in the rat spleen. Peptides 13:291-296.

26. Costerousse, O., Allegrini, J., Lopez, M., and Alhenc-Gelas, F. 1993. Angiotensin I-converting enzyme in human circulating mononuclear cells: genetic polymorphism of expression in T-lymphocytes. Biochem J 290 (Pt 1):33-40.

27. Weinstock, J. V., and Kassab, J. T. 1984. Angiotensin II stimulation of granuloma macrophage phagocytosis and actin polymerization in murine schistosomiasis mansoni. Cell Immunol 89:46-54.

28. Weidanz, J. A., Wittman, V. P., Lee, K., and Thekkumkara, T. J. 2002. A Functional Role for Angiotensin Type I Receptor in Stimulated T Lymphocytes. In Experimental Biology 2002. V. T. Marchesi, editor. New Orleans, La.: Federation of American Societies for Experimental Biology. A697-A698.

29. Femandez-Castelo, S., Arzt, E. S., Pesce, A., Criscuolo, M. E., Diaz, A., Finkielman, S., and Nahmod, V. E. 1987. Angiotensin II regulates interferon-gamma production. J Interferon Res 7:261-268.

30. Kristof, A. S., Marks-Konczalik, J., Billings, B., and Moss, J. 2003. Stimulation of STAT1-dependent gene transcription by lipopolysaccharide and interferon-gamma is regulated by mammalian target of rapamycin. J Biol Chem. 278:33637-44.

31. Peilot, H., Rosengren, B., Bondjers, G., and Hurt-Camejo, B. 2000. Interferon-gamma induces secretory group IIA phospholipase A2 in human arterial smooth muscle cells. Involvement of cell differentiation, STAT-3 activation, and modulation by other cytokines. J Biol Chem 275:22895-22904.

32. Horiuchi, M., Hayashida, W., Akishita, M., Tamura, K., Daviet, L., Lehtonen, J. Y., and Dzau, V. J. 1999. Stimulation of different subtypes of angiotensin II receptors, AT1 and AT2 receptors, regulates STAT activation by negative crosstalk. Circ Res 84:876-882.

33. Horiuchi, M., Hayashida, W., Akishita, M., Yamada, S., Lehtonen, J. Y., Tamura, K., Daviet, L., Chen, Y. E., Hamai, M., Cui, T. X., et al. 2000. Interferon-gamma induces AT(2) receptor expression in fibroblasts by Jak/STAT pathway and interferon regulatory factor-1. Circ Res 86:233-240.

34. Lighvani, A. A., Frucht, D. M., Jankovic, D., Yamane, H., Aliberti, J., Hissong, B. D., Nguyen, B. V., Gadina, M., Sher, A., Paul, W. E., et al. 2001. T-bet is rapidly induced by interferon-gamma in lymphoid and myeloid cells. Proc Natl Acad Sci USA 98:15137-15142.

35. Alexander, W. S., Starr, R., Metcalf, D., Nicholson, S. E., Farley, A., Elefanty, A. G., Brysha, M., Kile, B. T., Richardson, R., Baca, M., et al. 1999. Suppressors of cytokine signaling (SOCS): negative regulators of signal transduction. J Leukoc Biol 66:588-592.

36. Kile, B. T., and Alexander, W. S. 2001. The suppressors of cytokine signaling (SOCS). Cell Mol Life Sci 58:1627-1635.

37. Khaper, N., and Singal, P. K. 2001. Modulation of oxidative stress by a selective inhibition of angiotensin II type I receptors in MI rats. J Am Coll Cardiol 37:1461-1466.

38. Bayorh, M. A., Ganafa, A. A., Socci, R. R., Eatman, D., Silvestrov, N., and Abukhalaf, I. K. 2003. Effect of losartan on oxidative stress-induced hypertension in Sprague-Dawley rats. Am J Hypertens 16:387-392.

39. Wamholtz, A., Nickenig, G., Schulz, B., Macharzina, R., Brasen, J. H., Skatchkov, M., Heitzer, T., Stasch, J. P., Griendling, K. K., Harrison, D. G., et al. 1999. Increased NADH-oxidase-mediated superoxide production in the early stages of atherosclerosis: evidence for involvement of the renin-angiotensin system. Circulation 99:2027-2033.

40. Yanagitani, Y., Rakugi, H., Okamura, A., Moriguchi, K., Takiuchi, S., Ohishi, M., Suzuki, K., Higaki, J., and Ogihara, T. 1999. Angiotensin II type 1 receptor-mediated peroxide production in human macrophages. Hypertension 33:335-339.

41. Baker, R. J., Hemandez-Fuentes, M. P., Brookes, P. A., Chaudhry, A. N., Cook, H. T., and Lechler, R. I. 2001. Loss of direct and maintenance of indirect alloresponses in renal allograft recipients: implications for the pathogenesis of chronic allograft nephropathy. J Immunol 167:7199-7206.

42. Waaga, A. M., Gasser, M., Kist-van Holthe, J. E., Najafian, N., Muller, A., Vella, J. P., Womer, K. L., Chandraker, A., Khoury, S. J., and Sayegh, M. H. 2001. Regulatory functions of self-restricted MHC class II allopeptide-specific Th2 clones in vivo. J Clin Invest 107:909-916.

43. Shimizu, A., Yamada, K., Sachs, D. H., and Colvin, R. B. 2002. Mechanisms of chronic renal allograft rejection. II. Progressive allograft glomerulopathy in miniature swine. Lab Invest 82:673-686.

44. Albrecht, E. W., Stegeman, C. A., Tiebosch, A. T., Tegzess, A. M., and van Goor, H. 2002. Expression of inducible and endothelial nitric oxide synthases, formation of peroxynitrite and reactive oxygen species in human chronic renal transplant failure. Am J Transplant 2:448-453.

45. Libby, P., and Pober, J. S. 2001. Chronic rejection. Immunity 14:387-397.

46. Krivosikova, Z., Dusinska, M., Spustova, V., Sebekova, K., Blazicek, P., Heidland, A., and Dzurik, R. 2001. DNA damage of lymphocytes in experimental chronic renal failure: beneficial effects of losartan. Kidney Int Suppl 78:S212-215.

1. A method of inhibiting production of IFN-Γ in patients having a transplanted organ, the method comprising administering to the patient an amount of an angiotensin receptor-blocking compound, wherein the amount is effective to inhibit production of IFN-Γ by T cells.

2. The method of claim 1, wherein the angiotensin receptor-blocking compound is a type 1, angiotensin II receptor-blocking compound.

3. The method of claim 2, wherein the type 1, angiotensin II receptor-blocking compound is selected from the group consisting of losartan, valsartan, candesartan, and telmisartan.

4. A method of preventing inflammation in patients having a transplanted organ, the method comprising administering to the patient an amount of an angiotensin receptor-blocking compound, wherein the amount is effective to prevent macrophage activation.

5. A method of reducing intragraft inflammation in patients having a transplanted organ, the method comprising administering to the patient an amount of an angiotensin receptor-blocking compound, wherein the amount is effective to inhibit production of IFN-Γ by T cells.

6. The method of claim 5, wherein the transplanted organ is a kidney.

7. A method of inhibiting production of IFN-Γ in patients having a transplanted kidney, the method comprising administering to the patient having a transplanted kidney an amount of an angiotensin receptor-blocking compound, wherein the amount is effective to inhibit production of IFN-Γ by T cells.

8. The method of claim 7, the angiotensin receptor-blocking compound is a type 1, angiotensin II receptor-blocking compound.

9. The method of claim 6 wherein the type 1, angiotensin II receptor-blocking compound is selected from the group consisting of losartan, valsartan, candesartan, and telmisartan.

10. A method of treating chronic allograft nephropathy, the method comprising administering to a patient having, or suspected of having, chronic allograft nephropathy (CAN) an anti-CAN-effective amount of an angiotensin receptor-blocking compound effective to inhibit T-cell production of IFN-Γ.

11. The method of claim 10, wherein the angiotensin receptor-blocking compound is a type 1, angiotensin II receptor-blocking compound.

12. The method of claim 11, wherein the type 1, angiotensin II receptor-blocking compound is selected from the group consisting of losartan, valsartan, candesartan, and telmisartan.
